(19) United States
(12) Patent Application Publication
(10) Pub. No.: US 2023/0355832 A1
(43) Pub. Date: Nov. 9, 2023

(54) SYSTEM AND METHOD FOR INTEGRATED ENDOLUMINAL EMBOLIZATION AND LOCALIZED DRUG DELIVERY

(71) Applicants: Said Kamal Farha, Pleasantville, NY (US); Carlos Franco Pujante, Zurich (CH); Markus Grob, Reinach (CH); Salvador Pane Vidal, Zurich (CH); Kamal FARHA, Pleasantville, NY (US); Bradley Nelson, Zumikon (CH); Mark Farha, Ann Arbor, MI (US)

(72) Inventors: Said Kamal Farha, Pleasantville, NY (US); Carlos Franco Pujante, Zurich (CH); Markus Grob, Reinach (CH); Salvador Pane Vidal, Zurich (CH); Kamal FARHA, Pleasantville, NY (US); Bradley Nelson, Zumikon (CH); Mark Farha, Ann Arbor, MI (US)

(21) Appl. No.: 18/025,820

(22) PCT Filed: Sep. 16, 2021
(86) PCT No.: PCT/US2021/050686

§ 371 Date: Mar. 10, 2023

Related U.S. Application Data
(60) Provisional application No. 63/079,340, filed on Sep. 16, 2020.

(51) Int. Cl. A61L 24/00; A61K 49/18; A61M 25/01; A61M 25/00; A61B 18/14; A61B 18/00

(52) U.S. Cl. CPC A61L 24/0094; A61L 24/0042; A61L 24/0031; A61K 49/18; A61M 25/0127; A61M 25/0026; A61B 18/1492; A61L 24/001; A61M 2025/0042; A61L 2400/06; A61L 2430/36; A61L 2300/442; A61B 2018/00577

(57) ABSTRACT

A method for embolization of a blood vessel in a body, wherein at least one gelling component is biocompatible, biodegradable, with radiopaque capability and in liquid form to be supplied to a blood vessel by means of a single or multi lumen microcatheter and forming once in contact with a gelling agent in situ a deformable solid matrix in the body. The microcatheter may be provided with a first lumen disposed inside a second lumen. The gelling agent may be supplied to the blood vessel before the gelling component. Therapeutic compositions may be supplied to the blood vessel through the microcatheter.

### CROSS-REFERENCE TO RELATED APPLICATIONS

This application claims the benefit of U.S. Provisional Patent Application Ser. No. 63/079,340 filed Sep. 16, 2020, the disclosure of which is incorporated herein by reference.

### BACKGROUND

### Field of the Invention

This disclosure is in the field of systems and methods for embolization of blood vessels. This disclosure is also in the field of systems and methods for embolization of blood vessels with integrated drug delivery methods.

### Description of the Related Art

Embolization refers to the passage and lodging of an embolus within the bloodstream. It may be of natural origin (pathological), in which sense it is also called embolism, for example a pulmonary embolism. It also may be artificially induced for therapeutic reasons, as a hemostatic treatment for bleeding or as a treatment for some types of cancer by deliberately blocking blood vessels to starve the tumor cells.

The materials currently used for embolization have significant shortcomings. They include steel coils, acrylamides, various kinds of microspheres and other small particles. Current embolization materials trigger bodily reaction to a foreign substance or sub-optimal tissue ingrowth unable to withstand intra-vessel blood pressure

Current embolization materials trigger a bodily reaction to a foreign substance or sub-optimal tissue ingrowth unable to withstand intra-vessel blood pressure. Substances such as steel coils and acrylamides are non-resorbable materials, which the body treats as foreign and reacts to their intra-arterial application—leading to the early formation of collateral blood vessels that defeat the purpose of the embolization. Other embolizing agents, such as degradable starch microspheres, collagen microspheres, polylactic acid microspheres, iodized oil, resin particles or gelatin particles, are resorbable, do not offer optimal tissue ingrowth and are unable to adhere strongly to the intimal layer of the vessel. Furthermore, due to their particle size, they hardly reach arterioles having a diameter below 40 μm. This was found to be a major disadvantage, since non-resorbable microspheres with diameters of 15 μm or 32.5 μm (in contrast to 50 μm) were found to selectively lodge in malignant tissue than into the surrounding normal hepatic parenchyma. The optimal embolization material will be fully degraded only after sufficient time has been allowed for tissue ingrowth and the tumor tissues has been adequately starved of nutrients carried by blood in order to induce necrosis. This time frame is typically thought to be less than 30 days.

The use of embolization for cancer management involves a multi-pronged approach in which the embolus, besides blocking the blood supply to the tumor, also often includes an ingredient to attack the tumor chemically or with irradiation. When it bears a chemotherapy drug, the process is called chemoembolization. Transcatheter arterial chemoembolization (TACE) is the usual form. When the embolus bears a radiopharmaceutical for unsealed source radiotherapy, the process is called radioembolization or selective internal radiation therapy (SIRT).

In the treatment of hepatocellular carcinoma, embolization of hepatic arteries via TACE is indicated in patients with intermediate stage disease (BCLC stage B) who have large or multi-nodular disease without portal vein invasion or extrahepatic metastasis. TACE is contraindicated in patients with poor liver function (decompensated cirrhosis) or portal vein thrombosis.

Current embolization methodologies do not integrate response prediction or response monitoring. A major challenge with TACE and other locoregional therapies is the lack of reliable tools to determine which patients are good candidates for treatment who are likely to respond. Considering the highly malignant and difficult to treat nature of hepatocellular carcinoma, marked by a ˜70% 5-year recurrence rate after initial treatment with curative intent, there has been ongoing investigation into selection of the right patient for the right procedure, and early response monitoring to determine if treatments have been effective. With respect to TACE, pre-treatment genomic biomarkers to predict response have been described, and a patent for the genetic signature exists. Additionally, levels of serum biomarkers including DCP, HIF-1A, VEGF, and LDH have been demonstrated to be predictive of response when measured ˜1 month post treatment5. Imaging biomarkers of TACE response are an emerging area of investigation, both in the intra-procedural setting (TRIP-MRI) and in the post-procedural setting by measurement of apparent diffusion coefficient and decreases in venous enhancement at 1 month after the procedure.

Embolization material that is detectable via imaging is central to this solution. There is only one preliminary report of an embolic agent which incorporates radiopaque elements. Maurer, Hepatic artery embolization with a novel radiopaque polymer causes extended liver necrosis in pigs due to the occlusion of the concomitant portal vein, Journal of Hepatology 2000; 32; 261-268, investigated the use of a radiopaque polyurethane termed DegraBloc® to overcome several disadvantages inherent to the previously used materials (“Hepatic artery embolization with novel radiopaque polymer causes extended liver necrosis in pigs due to occlusion of the concomitant portal vein”, Journal of Hepatology 2000; 32: 261-268). The disclosure of this article is herewith fully incorporated by reference. All other embolization materials, apart from iodized oil, have to be mixed and hence diluted with conventional radiopaque materials, e.g. with polyurethane-based plastic X-ray contrast materials that have been described by Neuenschwander et al. in U.S. Pat. No. 5,319,059, which is herewith also fully incorporated by reference. To facilitate post-embolization response monitoring, integration of embolization material and an MRI contrast agent presents an exciting opportunity. There are a number of pre-clinical cancer models leveraging nanoparticle scaffolds outfitted with polyethyeneimine and MRI contrast or fluorescence based optical imaging media8-12, but there is no commercially available embolization material which incorporates MRI contrast.

Application of the embolization polymer using existing techniques requires a high degree of specificity to avoid unintended necrosis of healthy tissue. For example, the polyurethane DegraBloc® was used by Maurer et al. as an alcoholic solution of a block copolymer. As soon as an aqueous solution, e.g. blood, or a water-containing surface came into contact with the liquid polyurethane, a precipitation process started and finished within 3-5 min, depending on the size of the occluded vessel. The result was a solid, elastic intravascular cast. Experimental pancreatic duct occlusion showed that the polymer was fully biodegradable and disappeared from the duct within 14 days.

It was found that the intra-arterial applicability of the polyurethane was excellent and that DegraBloc® did not produce any obvious systemic side effects in pigs, as long as the vessels supplying the stomach and gallbladder were not accidentally embolized too. Also, it was demonstrated that the initially liquid DegraBloc® was able to pass into the most peripheral arteries where it became solid upon contact with the arterial wall and with blood. Further tests showed that the polyurethane DegraBloc® is a biocompatible, slowly degradable, radiopaque embolic agent capable of incorporating adriamycyn (doxorubicin), a well-known anti-tumor agent.

The main drawback of the method described by Maurer et al., however, is the fact that the position of precipitation, and thus of embolization, cannot be reliably controlled. As mentioned above, a contraindication to TACE is concurrent portal vein thrombosis. In this scenario, occlusion of both arterial and venous blood supply will cut off all blood flow to the liver and lead to widespread liver necrosis. This was seen in several pigs in the pre-clinical model DegraBloc® was trialed on in Maurer. In order to avoid this complication, control of embolization material and specific application to the vessel of interest is integral.

Another shortcoming of existing techniques for embolization is use as a delivery system for therapeutic drugs. TACE enables delivery of anti-neoplastic chemotherapies, however, such localized delivery of other types of therapeutics does not yet exist The major principle behind TACE is capitalizing on the synergy between embolization of the vessels supplying the tumor to starve the cancer cells of necessary nutrients to fuel their overactive machinery while also providing cytotoxic chemotherapeutic agents targeting and disrupting key cancer cell functions, ultimately leading to a double hit against cancer cells and their eventual death.

It would be highly desirable for the embolization material to carry and release therapeutic agents targeting cancer cells. It would be highly desirable to deliver both small molecule chemotherapies, and antibody therapies targeting the immune system (“immunotherapies”) or blood vessel formation (“VEGF”). However, delivery systems are needed not just for anti-cancer therapies, but also for gene therapies and other nucleotide therapeutics as well as anti-viral drugs.

### SUMMARY OF THE INVENTION

In various embodiments, the invention comprises a method of embolizing a blood vessel by supplying a gelling component and a gelling agent to the blood vessel through a multi-lumen catheter. Additional compositions may be supplied to the blood vessel through the multi-lumen catheter, including therapeutic agents and MRI contrast agents. The gelling agent may be started before the gelling component to prevent premature reaction of the gelling component to form an embolus. In some embodiments the multi-lumen catheter has a first lumen disposed inside a second lumen. In some embodiments the second lumen completely surrounds the first lumen. In some embodiments the multilayer catheter is designed with a microfluidics regime to enhance mixing and reactivity of the hydrogel formation just before release to the blood stream.

### BRIEF DESCRIPTION OF THE DRAWINGS

FIG. 1 is a cross-sectional view of an embodiment of the multi-lumen catheter of the present invention.

FIG. 2 is a cross-section view of another embodiment of the multi-lumen catheter of the present invention.

FIG. 3 is a schematic representation of a partially embolized blood vessel.

FIG. 4A depicts a steerable micro-cathether in a first configuration.

FIG. 4B depicts a steerable micro-cathether in a second configuration.

FIG. 4C depicts a steerable micro-cathether in a third configuration.

FIG. 4D depicts a steerable micro-cathether in a fourth configuration.

FIG. 4E depicts a steerable micro-cathether in a fifth configuration.

FIG. 4F depicts a steerable micro-cathether in a sixth configuration.

### DETAILED DESCRIPTION

In view of the above, it is thus an object of the present invention to provide an improved method for blood vessel embolization, which in particular allows for an exact control of the precipitation position and thus of the embolization position, yet at the same time allows for controlled delivery of therapeutic substances to the target site. This problem is solved by the method according to claim 1 and claim 2. Preferred embodiments are subject of the dependent claims. The present invention relates to a method for blood vessel embolization, in a body, wherein at least one biocompatible and biodegradable gelling component in liquid form is supplied to a blood vessel. Within the context of the present invention the term “in liquid form” stands for a solution, emulsion, suspension, and the like. Preferably, the gelling agent is dissolved or suspended in water, ethanol or DMSO or a mixture thereof.

Then at least one gelling component is supplied by means of a single or multi lumen microcatheter and forms in situ a deformable solid matrix in the body. As regards the term “microcatheter” it is noted that in some prior art documents, e.g. U.S. Pat. Nos. 6,254,588 B1, 8,992,506 B2, US 2015 0005801 A1 and U.S. Pat. No. 5,919,171 A, said term is limited to a catheter with an outer diameter of 0.3-1 mm. However, the term microcatheter as used throughout this application shall not be understood in this limiting fashion and shall also encompass catheters with an outer diameter between 0.3-3 mm.

The deformable solid matrix is flexible and elastic. In addition, it is able to deform and then regain its shape and structural integrity. Most preferably, the deformable solid matrix adapts to the shape of the blood vessel. Such polymer systems are the subject of abundant research and development activities. The polyurethane based polymer—Degrabloc—has already been described above. Additionally, Adherus from Stryker, a dural sealant used in neurosurgical applications, is a polyethyleneimine/polyethylene glycol based hydrogel delivered via a two chamber applicator, and VIVO surgical sealant from Adhesys medial, a polyurethane and isocyanine based surgical sealant with an amino based curing agent to facilitate quick setting time intended to be used in vascular surgery, are examples of such materials. The present invention is intended to build on these existing solutions and address key gaps.

If a polyurethane material is supplied to the blood vessel, it is formed from at least one diol precursor compound and at least one diisocyanate precursor compound.

An X-ray or MRI contrast material is bound to the at least one gelling component. Preferably, said X-ray or MRI contrast material is preferably bound to the polyurethane material. Said X-ray or MRI contrast material is preferably covalently bound however, an ionic bond or any other chemical bond is possible too. The X-ray or MRI contrast material bound to the polyurethane material and/or to the hydrogel precursor provides the latter with radiopaque characteristics, i.e. it will be visible in an X-ray or MRI. In case of a hydrogel said X-ray or MRI contrast material is preferably electrostatically bound to or precipitated into the hydrogel.

The at least one gelling component is such that it forms in situ a deformable solid matrix in the body. Preferably, the deformable solid matrix is a hydrogel or a polyurethane matrix. Hydrogel formation generally occurs through a chemical reaction that is capable of being initiated by several ways, as disclosed on many occasions in the art: For instance, US 2003 0134032 A1 discloses a method of initiating the formation of a hydrogel in situ by delivering an initiator and a gellable composition, that forms a hydrogel in response to the initiator, to the intended site of formation of a hydrogel. WO 2001/028031 discloses the in-situ formation of a bioadhesive hydrogel. US 2017 0313828 A1 discloses a method of forming a dendrimer hydrogel comprising one or more amine end-functioned polyamidoamine as a first reactant and one or more small molecule, polymer, hyperbranched molecule or dendrimer as a second reactant, wherein the second reactant comprises one or more acrylate groups and wherein the first and the second reactant reacting by way of conjugate addition. WO 2018/009839 A1 discloses a method for providing intracavitary brachytherapy, delivery of a thiol-Michael addition hydrogel to a body cavity, expanding the thiol-Michael addition hydrogel, and displacing tissue and/or organs by the expanding thiol-Michael addition hydrogel.

Within the context of the present invention the term “deformable solid matrix” stands for an object preferably a tissue, layer or matrix that is in a solid state. A solid state is here defined as a state that is not liquid or gaseous. The term deformable is defined here as a state where the solid matrix is elastic or plastic deformable under a certain force.

In case of the present invention, the hydrogel formation of the gelling component or gelling agent is preferably initiated through contact with the gelling agent, preferably body fluids (e.g. blood, water, plasma, etc.). As such, the gelling component is able to be supplied through the microcatheter to the desired location (i.e. the target site) and once released from the catheter it will come into contact with body fluids and therefore form the hydrogel. However, it is also contemplated that the gelling component or several gelling components, in some embodiments, will form a hydrogel by itself at the target site. This could be implemented by a two-lumen microcatheter, which delivers two gelling components, one in each lumen, (or a gelling component and a gelling agent) to a target site, where they react together and form a hydrogel. Preferably the hydrogel has a short gelling time that results in an instant and quick formation of the hydrogel.

The function of the hydrogel in the method of the present invention is in some embodiments two-fold. On the one hand, the hydrogel is in some embodiments used to cause embolization of the blood vessel. On the other hand, incorporation of a drug into a hydrogel that is biodegradable within the body is able to be used for controlled, targeted drug application. Specific agents that could be incorporated into the system include small molecule, protein, and nucleotide therapeutics with mechanisms of action including but not limited to “anti-angiogenic”, “immunotherapy”, as well as nucleotides including plasmid DNA and siRNA.

Furthermore, the system is not limited to the incorporation of anti-cancer agents. Hepatitis is an incredibly common infectious cause of liver inflammation, with the WHO estimating a prevalence of ˜250 million cases of Hepatitis B and ˜70 million cases of hepatitis C in 2015. Hepatitis is a vaccine preventable illness, and hydrogels have been explored as a delivery mechanism to deliver Hepatitis B surface antigen in order to stimulate immune response and increase vaccine efficacy in the 5-10% of vaccine non-responders. Furthermore, hydrogels have also proven useful in hepatitis C treatment by limiting the degradation and prolonging the half-life of PEG-ylated interferon, an important hepatitis C treatment. Other PEG-ylated protein therapeutics have been investigated in the setting of hydrogel delivery. Specifically, the pharmaceutically active agent is able to be provided together with the gelling component though the microcatheter to the tumor site, whereby formation of the hydrogel will lead to encapsulation of the active agent. Degradation of the hydrogel will then lead to sustained release of the active agent, whereby the release is able to be controlled via the degradation rate of the hydrogel.

In some embodiments of the invention, the active agent is encapsulated in a virosome with fusion activity. The fusion of the virosome with a target cell, such as a cancer cell, releases the active agent into the target cell. In some embodiments the virosome incorporates PEG lipids in the virosome membrane, or the virosome can be carried into a PEG stream. The PEG lipids may be coupled to antibody molecules that are specific to cancer or other target cells. The PEG coat inhibits the normal HA affinity to sialic acid which reduces the virosome affinity to non-target cells. The PEG coat of the virosome makes it compatible with encapsulation in a PEG hydrogel. After creation of a hydrogel embolus with encapsulated virosomes, the normal degradation of the matrix of the hydrogel results in a delayed release of the virosomes over time to provide extended delivery of active agent to the tumor or other treatment site.

The polyurethane material and the PEI/PEG hydrogels or other hydrogels mentioned in connection with the instant invention—if supplied to the blood vessel—preferably also has two functions: facilitating detection by an imaging modality and being preferably capable of precipitation to cause an embolization at a predetermined location in the blood vessel. Thanks to its visibility via the incorporation of contrast material or fluorescence dye, the exact location of the embolization is able to be verified by imaging modalities including but not limited to X-Ray, CT, MRI, Ultrasound or fluorescence imaging. Precipitation of the polyurethane material is preferably caused by coming into contact with the gelling agent. The nature of body tissues is such that they act as an anionic substrate that has the propensity to interact strongly with cationic polymers, enabling hydrogel precursor and or the polyurethane material to act as a suitable solution to fill cavities or repair damaged tissues. This is especially relevant in the case of an aneurysm, in which blood vessel diameter increases in size as a result of weakening of the vessel wall, or a laceration, in which the blood vessel wall is damaged such that there is a connection with the environment outside the vessel, allowing blood to leak out. It is plausible that the hydrogel precursor and or the polyurethane material are delivered to a site of an aneurysm or laceration to stabilize the blood vessel wall until these defects are able to be fixed with an operation.

In case a multi lumen catheter is used, the gelling agent is able to be supplied via a first lumen and at least one gelling component is able to be supplied via a second lumen. This allows to simultaneously supply both the gelling agent and the at least one gelling component to the desired embolization location, and to immediately contact the gelling agent with the at least one gelling component as soon as they are released from the catheter, enabling exact control of the precipitation position, without any shift in the position of the gelling component prior to precipitation. Consequently, it is possible to restrict embolization to the desired vessels, and the risk of causing necrosis to healthy tissues and resultant liver failure is significantly reduced.

Preferably, the deformable solid matrix prepared from the gelling component will generally perfectly fit into its surroundings, in this case the walls of the blood vessel, and thus completely fill up the space within the artery. The blockade is enhanced by the tissue ingrowth facilitated by the strong interaction between the cationic matrix and anionic tissue substrate (vessel wall).

The polyurethane used in the method of the present invention preferably is able to be pre-formed outside the patient's body. The PEI/PEG hydrogel formation can be started in the microfluidic section of the catheter and completed in the blood vessel.

The method of the present invention is able to be used to fully or partially embolize the blood vessel.

The microcatheter is able to be treated with a hydrophobic material like Teflon to facilitate movement within the body, in particular a blood vessel. In a preferred embodiment the microcatheter “iVascular” from Boston scientific is used to deliver components to the target site.

In a preferred embodiment, a microcatheter is used, whereby a gelling agent is supplied by the first lumen and the therapeutic agent and the at least one gelling component are provided via the second lumen, whereby the gelling agent and the at least one gelling component form a deformable solid matrix after contact with each other. This allows a homogeneous distribution of the therapeutic agent within the deformable solid matrix.

If separate supply of additional components is desired, a microcatheter with three or more lumen is used in some embodiments. For instance, in some embodiments a third lumen is used for supplying a therapeutic agent, whether that is an anti-viral or anti-neoplastic small molecule, protein, or nucleotide therapeutic.

In a preferred embodiment, a three lumen microcatheter is used, whereby the gelling agent, the gelling component and the therapeutic agent are all provided via separate lumen. In this case, once released from the microcatheter, the gelling agent and the gelling component will form a deformable solid matrix, containing the therapeutic agent within. The deformable solid matrix is able to thereby have different degradation rates based on proximity to the tumor, with the matrix component closest to the tumor possessing the quickest degradation rate in order to expedite delivery of the therapeutic cargo.

Alternatively, as mentioned, the gelling component is able to also be delivered in a single lumen catheter in a combination with a therapeutic agent and form the deformable solid matrix with the therapeutic agent embedded therein.

The gelling component is able to also react in combination with another gelling agent or a plurality of the gelling agents or a body fluid such as water or blood to form the hydrogel. The degradation rate of the deformable solid matrix in water or blood at 37° C. is preferably between 1 to 5 days. The degradation rate can be accelerated by introducing enzymes like pancreatic enzymes through independent Lumen that can directly interact with the gelled material and degrade it. The byproduct of such enzymatic degradation can be sucked out by reversing the flow direction of the delivering lumen.

The hydrogel precursor is preferably selected from a group consisting of gelatin, chitosan, heparin, cellulose, dextran, dextran sulfate, chondroitin sulfate, keratan sulfate, dermatan sulfate, alginate, collagen, albumin, fibronectin, laminin, elastin, vitronectin, hyaluronic acid, fibrinogen, multi-arm-polyethyleneglycol, a tetronic series (4-arm-PPO-PEO), and a combination thereof, said multi-arm-polyethyleneglycol being selected from among 3-arm-polyethyleneglycol (3arm-PEG), 4-arm-polyethyleneglycol (4arm-PEG), 6-arm-polyethyleneglycol (harm-PEG), 8-arm-polyethyleneglycol (8arm-PEG), phenol derivate, aniline derivate, dopa derivate, polycationic polymer linker, polyanionic polymer linker, polyamphoteric polymer linker, polynonionic polymer linker, polyester linker, polyanhydride linker, polyorthoester linker, polyurethane linker, polyamide linker, polypeptide linker, polyaliphatic linker, polyaromatic linker, polyethylene glycol(PEG)-polylactic acid (PLA) linker, polyethylene glycol(PEG)-polycarpropactone (PCL) linker, polyethylene glycol(PEG)-poly(DL-lactic-co-glycolic acid) (PLGA) linker, poly((propylene)fumarate) linker, poly((ethylene)fumarate) linker, polyethyleneglycol (PEG) linker, polyethylene oxide (PEO) linker, polyethylenimine (PEI) linker, polypropylene oxide (PPO) linker, polyvinyl alcohol (PVA) linker, poly(N-isopropylacrylamide) (polyNIPAM) linker, polyfumarate, polyorganophosphazene linker, polyacrylic acid (polyAAc) linker, polyacrylsulfonate linker, poly hydroxyethylmethacrylate(PolyHEMA linker, PEO-PPO-PEO (Pluronic® series), 4-arm PEO-PPO-PEO (Tetronic® series), PEG-PEI, PEG-PVA, PEG-PEI-PVA, PEI-PVA, poly(NIPAAM-co-AAc), poly(NIPAAM-co-HEMA),polysaccharide, PAMAM G5-(NH2)16-(Ac)112, PAMAM G5-(NH2)22-(Ac)106, PAMAM G5-(NH2)38-(Ac)90, PAMAM G5-(NH2)64-(Ac)64, PAMAM G5-(NH2)128, PEG comprising one or more acrylate groups, polyethylene imine, and combinations thereof. More preferably, the hydrogel comprises dextran and chitosan (e.g. Endomedix Biopolymer Technology).

The polyurethane material used in the method of the present invention is, in various embodiments, prepared from one or several different diol precursor compounds and from one or several different diisocyanate precursor compounds.

In a preferred embodiment, at least part of the diol precursor compound(s) is selected from the group consisting of a glycerine monoester of diatrizoic acid (1), a glycerine monoester of a triiodobenzoic acid derivative (2, 3, 4, 5), and an iodinated pyridon-4 derivative (6):

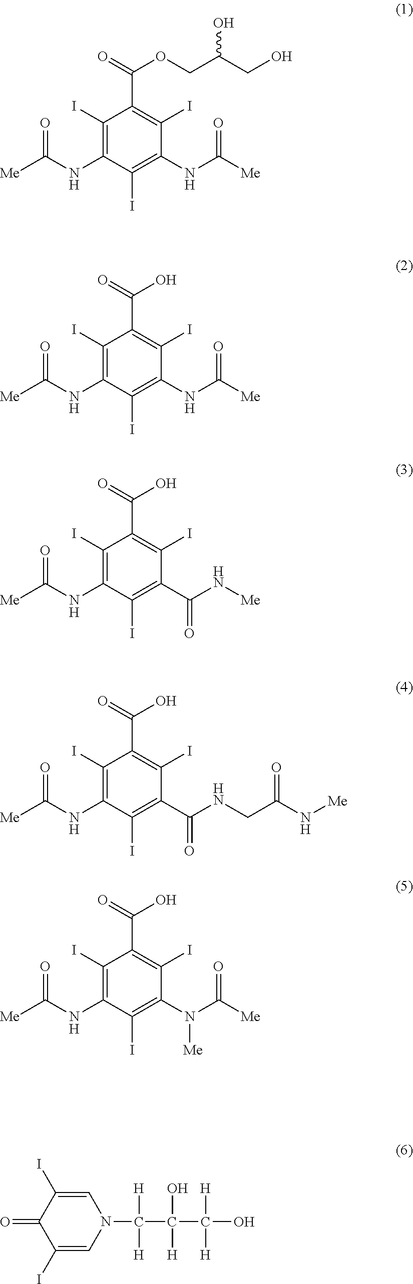 [SMILES: CC(=O)Nc1c(I)c(NC(C)=O)c(I)c(C(=O)OCC(O)CO)c1I ; CC(=O)Nc1c(I)c(NC(C)=O)c(I)c(C(=O)O)c1I ; CNC(=O)c1c(I)c(NC(C)=O)c(I)c(C(=O)O)c1I ; CNC(=O)CNC(=O)c1c(I)c(NC(C)=O)c(I)c(C(=O)O)c1I ; CC(=O)Nc1c(I)c(C(=O)O)c(I)c(N(C)C(C)=O)c1I ; O=c1c(I)cn(CC(O)CO)cc1I]

Thus, the polyurethane material used in the method of the present invention is preferably prepared from at least one of the diol precursor compounds (1-6), whereby said compound (1-6) is, in some embodiments, the only diol precursor compound used or there are, in other embodiments, one or more other diol precursor compounds used, which in some embodiments are optionally also selected from the diol precursor compounds (1-6).

According to a preferred embodiment, at least one diol selected from the group consisting of
bi-valent alcohols of the type

HO—(CH₂)ₙ—OH n=2-12
polyether diols of the type

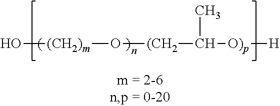

polyester diols on the basis of:
adipic acid/ethylene glycol-co-propylene glycol, adipic acid/ethylene glycol, adipic acid/propylene glycol;
polyglycol diol and polyglycol-co-lactide diol;
poly-3-hydroxybuyric acid diol and poly-3-hydroxybutyric acid-co-3-hydroxy valeric acid diol;
poly-3-hydroxy valeric acid diol;
poly-caprolactone diol;
de-polymerized cellulose; or
de-polymerized cellulose acetate;
is used as a co-condensible diol compound in the preparation of the polyurethane material used in the method of the present invention.

The polyester diols on the basis of II, III and IV are preferably produced by transesterification of higher molecular polyesters with ethylene glycol, diethylene glycol and triethylene glycol with simultaneous cleavage into a plurality of macro-diols having a mean molecular weight between about Mn 500 and 10,000.

According to a preferred embodiment, at least part of the diisocyanate precursor compound is selected from the group consisting of:
a. 5-isocyanato-1-(isocyanatemethyl)-1,3,3-tri-methylcyclohexane(IDPI)
b. 1,3-bis(1-isocyanato-1-methyl)-benzene (TMXDI)
c. hexamethylene diisocyanate (HDI)
d. 2,2,4-trimethyl-hexamethylene diisocyanate (THDI).

Thus, the polyurethane material used in the method of the present invention is preferably prepared from at least one of the diisocyanate precursor compounds (a-d), whereby said compound (a-d) is, in some embodiments, the only diisocyanate precursor compound used or there are, in other embodiments, one or more other diisocyanate precursor compounds used, which in some embodiments is optionally also selected from the diisocyanate precursor compounds (a-d).

Condensation of the diol and diisocyanate precursor compounds occurs in solution in a mixture of dioxane/dimethylformamide having a mixing ratio between 1:1 and 20:1 at temperatures between 40 and 100° C., with or without a catalyst. Isolation of the polyurethane material is accomplished by precipitation in water. Purification is accomplished by repeated dissolution of the polymer and precipitation in water. Thus, the polyurethane material is fully prepared outside the patient's body.

According to a particularly preferred embodiment, the polyurethane material used in the method of the present invention is represented by the formula (7):

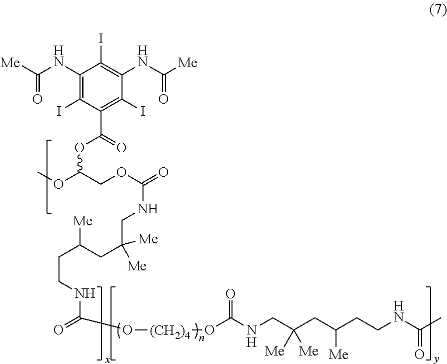

where x≥1, y≤1000, and 1≤n≤50. The synthesis of this polymer has been described by Maurer et al. in Journal of Hepatology 200; 32: 261-268. It has been found that the polyurethane of formula (7) is particularly well suited for controlled blood vessel embolization, and also allows for the incorporation anti-tumor agents or other drugs, which in some embodiments is then be released over time thanks to biodegradation of the polymer in the body.

A particularly preferred polyurethane material is commercially available under the tradename DegraBloc® and described in U.S. Pat. No. 5,319,059.

Differentiator—Specificity of Tumor Cell Targeting via Mechanical Delivery to Highly Specific site using microcatheter capable of reaching and blocking the small blood vessels and delivering the therapeutic load at the specific predetermined site.

The double or multi lumen catheter used in the method of the present invention preferably has an external diameter of about 0.5-2.0 mm, more preferably of 1.0-1.5 mm.

Preferably, the first lumen, which is used for supplying the gelling agent, is at least partially surrounded by the second lumen, which is used for supplying the at least one gelling component. Two possible arrangements of the two lumina within the catheter are schematically shown in FIGS. 1 and 2 showing a cross section of the catheter, with A being the first lumen and B being the second lumen. Particularly preferably, the first lumen A is completely surrounded by the second lumen B in the cross section (FIG. 2).

According to a preferred embodiment, the tip of the second lumen penetrates farther than the tip of the first lumen.

The term “penetrates farther” is defined here as closer to the target site.

According to a preferred embodiment, the release of the gelling agent is started earlier than the release of the at least one gelling component. This guarantees that, once the gelling component is released, it will immediately be in contact with the gelling agent and therefore cannot migrate prior to precipitation.

Furthermore, it is preferred that during the release of at least one gelling component, there is also a release of the gelling agent. Thus, the release of the gelling agent is preferably started prior to the release of the at least one gelling component and is not stopped before the release of the at least one gelling component is stopped. The gelling agent in some embodiments is still released after the release of the at least one gelling component has been stopped, if desired.

It is also possible to stop and restart the release of the at least one gelling component and/or the gelling agent several times, e.g. in order to switch the release position. But preferably, there should be no release of the at least one gelling component without simultaneously releasing the gelling agent.

Preferably the microcatheter is similar to catheters for radiofrequency ablation that include variable stiffness segments and a magnetic tip and/or is attached to a steerable micro-robot. The steerable nature of the catheter improves control and reduces the risk of inadvertent injury to bystander structures. A steerable micro-robot is able to be attached to the microcatheter and pull said catheter to the determined location. To remove the micro-robot from the patient's body the microcatheter is removed and so the attached micro-robot. The main limitation of remote magnetic navigation is that different magnetic fields cannot be applied at different magnet positions in the workspace. Therefore, a construction with variable stiffness segments enables a higher degree of control over the position of the catheter tip.

In a preferred embodiment the variable stiffness segments are based on a low melting point alloy and enable the tuning of stiffness and deformability of the tip of the catheter and that the magnetic tip of the catheter is able to be controlled by an external magnetic field. This construction enables a separate control over the variable stiffness segments and the magnetic tip. The variable stiffness segments are modified by conductive wires that induce heat into the segments to induce flexibility. The magnetic tip on the other hand is steered by magnetic fields, generate outside of the human body. The variable stiffness segments generating a torque on the tip that is negligible, since it is two orders of magnitude smaller than the one generate by the permanent magnet.

Preferably the magnetic field to steer the microcatheter and/or the micro-robot have a magnetic gradient of at least 0.1 T/m. The present invention is further illustrated by the following schematic figures:

While FIG. 2 shows a case were a first lumen A is completely surrounded by a second lumen C, FIG. 1 displays a case where the first lumen A is only partially surrounded by the second lumen C. As described above, the first lumen A is meant to be used for supplying the gelling agent and the second lumen C for supplying the at least one gelling component.

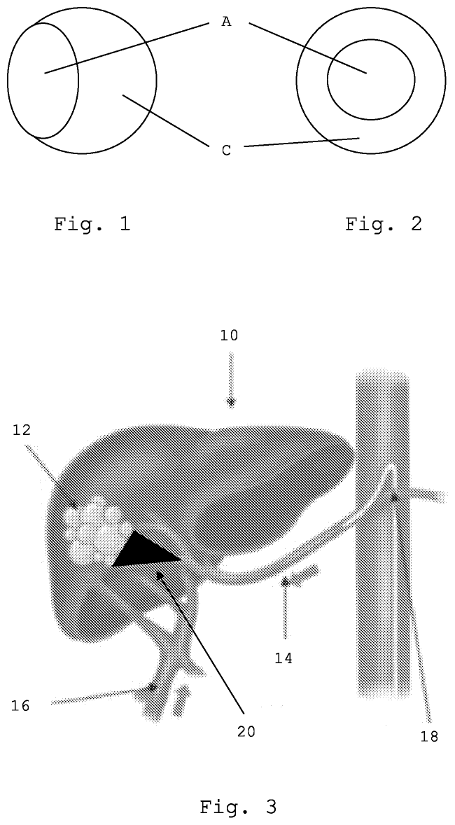

FIG. 3 shows a schematic representation of a partially embolized blood vessel: The liver 10 contains a tumor 12. While the tumor 12 is typically mainly supplied with blood from the blood vessel 14, the liver 10 itself mainly depends on the portal vein 16. Consequently, it is possible to selectively cut off the supply of the tumor 12 while the liver 10 is only weakly affected.

By means of a catheter 18, the gelling agent and at least one gelling component are supplied to the desired position in the hepatic artery 14, where the at least one gelling component is caused to precipitate and form blockade 20, effectively embolizing that part of the blood vessel 14.

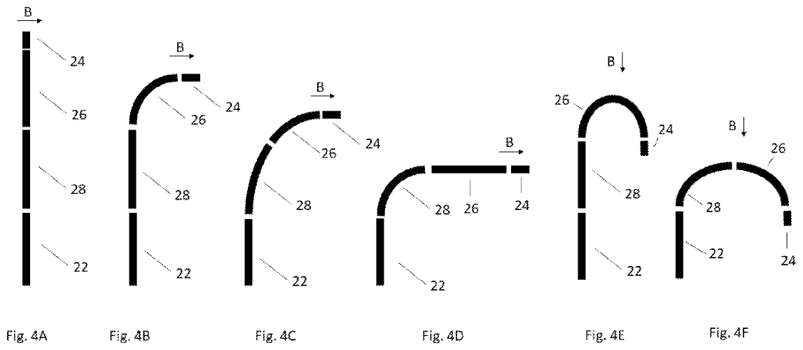

FIG. 4 shows a steerable catheter 22 interacting with a magnetic field B. A magnetic tip 24 followed by two variable stiffness segments 26, 28 are located at the top of the steerable catheter 22. The magnetic tip 24 interacts with the magnetic field B and tries to align its magnetic dipole. If as shown in FIG. 4A both variable stiffness segments 26, 28 are stiff, the magnetic tip 24 is not aligning its magnetic dipole to the magnetic field B and the tip 24 stays in the position. In FIG. 4B, an electric flow is induced in the first variable stiffness segment 26, while the second variable stiffness segment 28 stays stiff. The magnetic tip 24 aligns its dipole to the magnetic field B and the catheter 22 as a 90° turn with a short radius in its tip. In FIG. 4C electric flow is induced in both variable stiffness segments 26, 28 and allows the magnetic tip 24 to align its magnetic dipole to the magnetic field B and create a 90° turn with a longer radius. In FIG. 4D only the second variable stiffness segment 28 is soft, while the first variable stiffness segment 26 stays stiff. The magnetic tip 24 aligns its dipole with the magnetic field B and creates a 90° turn with a short radius. In FIG. 4E the direction of the magnetic field B is rotated 90° compared to FIG. 4A-D. Therefore, with the same setting as in FIG. 4B, the catheter has now a 180° turn with a short radius. In FIG. 4F the same magnetic field B orientation as in FIG. 4E but with a two soft variable stiffness segments 26, 28 create a 180° turn with a long radius.

The gelling agent used in some embodiments is pure water or any liquid composition comprising water, such as blood or an isotonic solution, for instance. According to a preferred embodiment, the gelling agent at least mainly consists of water or blood, with the blood preferably being the patient's own blood.

According to a preferred embodiment, the polyurethane material is provided in the form of an ethanolic solution, suspension or emulsion. This allows for a sufficient solubilization of the polyurethane material in order to enable the supply through the microcatheter. It is also possible to add small amounts of dimethylsulphoxide (DMSO) to the ethanolic polyurethane solution, suspension or emulsion, thereby improving the solubility. Preferably, a solution of 300 mg of the polyurethane per ml of a mixture of 93% ethanol and 3% DMSO is used. Solubility in some embodiments is further increased by heating, and it is also possible to use a more dilute solution.

The polyurethane material used in the method of the present invention, and in particular that of formula (7), is very well suited for use as a controlled release vehicle for active pharmaceutical agents. Additionally, integration of high molecular weight polyalkaleneimines offers enhanced delivery of negatively charged cargo (i.e. protein, nucleic acid therapeutics) to their target destination. Therefore, according to a preferred embodiment, one or more liposomes and/or therapeutic agents are supplied to the blood vessel. Depending on the solubility of said liposomes and/or therapeutic agents, it is preferred that they are supplied together with the at least one gelling component. Alternatively, it is possible to supply the liposomes and/or therapeutic agents separately, by means of a third lumen in the catheter.

By including a therapeutic agent in the at least one gelling component (solution) and/or the gelling agent or by supplying it separately, focused delivery to the tumor site is possible without affecting surrounding normal tissues. Since the therapeutic agent is trapped within the degradable matrix to a certain extent, the agent is able to be slowly released to impart it's effects over an extended period of time. This is especially important for drugs that classically have a short half-life. Thus, the method of the present invention allows for a controlled release of the therapeutic agent over a prolonged time (typically several days or weeks). Preferably, the degradation rate is able to be modulated by introducing a biodegrading agent (i.e. enzyme) to the target site to tightly control the degradation process, in particular the degradation rate. Such a biodegrading agent is able to be delivered together with the gelling component.

A therapeutic agent in some embodiments is supplied alone or in combination with a delivery system, in particular a targeted delivery system. For instance, an active pharmaceutical agent is, in some embodiments, encapsulated in liposomes, virosomes, exosomes, polymersomes, linear polymers or dendrimers or even in lipid material. Alternatively, in some embodiments, also pH sensitive compounds or Nano based materials are used. Agents that are able to be delivered in this manner span a broad scope of chemistries and mechanisms of action. They include but are not limited to cytotoxic chemotherapy falling into the classes of alkylating agents such as cisplatin (DDP) carboplatin (CBP), and oxaliplatin (L-OHP), nitrogen mustard, chlorambucil, cyclophosphamide (CTX), and ifosfamide (IFO), nitrosureas, such as N-methyl-N-nitrosurea (MNU), N′-[(4-amino-2-methylpyrimidin-5-yl)methyl]-N-(2-chloroethyl)-N-nitrosourea (ACNU), 1,3-bis(2-chloroethyl)-1-nitrosourea (BCNU), N-(2-chloroethyl)-N′-cyclohexyl-N-nitrosourea (CCNU), and N-(2-chloroethyl)-N′-(4-methylcyclohexyl)-N-nitrosourea (methyl CCNU); ethylenimines, such as 2,4,6-triethylene melamine compound (TEM) and thiotepa; methane sulfonates, such as busulfan, dacarbazine, procarbazine, plant alkaloids and anti-microtubule agents including vinblastine (VLB), vincristine (VCR), vindesine (VDS), navelbine (NVB), paclitaxel (PTX), and taxotere, anti-tumor antibiotics (intercalating agents) including anthracycline antitumor antibiotics, such as adriamycin (ADM; doxorubicin), daunorubicin (DNR), epirubicin (EPI or E-ADM), mitoxantrone (MTT, DHAD), and pirarubicin (THP); actinomycin antitumor antibiotics, such as actinomycin D (ACD); bleomycin antitumor antibiotics, such as bleomycin and pingyangmycin (A5); mitomycin antitumor antibiotics, such as mitomycin A, mitomycin B, and mitomycin C (MMC); mithramycin antitumor antibiotics, such a mithramycin (MTH) and olivomycin, other antibiotics, such as streptozotocin (STT), antimetabolites including thymidylate synthase inhibitors, such as 5-fluorouracil (5-FU), ftorafur (FT-207), tegadifur (difuradin FD-1), tegafur-uracil (UFT), and furtulon (5-DFUR); dihydrofolate reductase inhibitors, such as methotrexate (MTX), DNA polymerase inhibitors, such as cytarabine (Ara-c); ribonucleotide reductase inhibitors, such as hydroxyurea (HU), inosine dialdehyde, adenosine dialdehyde, and guanazole; purine nucleotide synthesis inhibitors, such as 6-mercaptopurine (6-MP) and andaminopterin, topoisomerase inhibitors, small molecule targeted therapies including but not limited to tyrosine kinase inhibitors such as imatinib, erlotinib, sunitinib, gefitinib, sorafenib, dasatinib, lapatinib, and nilotinib and the like, biologic therapies, including but not limited to such as ipilimumab, nivolumab, pembrolizumab and the like, and anti-viral drugs such as protease inhibitors, nucleotide/non-nucleotide polymerase inhibitors and interferons. Examples of suitable delivery systems are further described in Perche et al.: “Recent Trends in Multifunctional Liposomal Nanocarriers for Enhanced Tumor Targeting” (Journal of Drug Delivery, Volume 2013, Article ID 705265). This review article is herewith incorporated by reference with respect to suitable delivery systems.

Particularly preferably, adriamycin is supplied (ADM; doxorubicin). Adriamycin is the most popular agent used in TACE for hepatocellular carcinoma worldwide, and it is well soluble in DMSO and is typically administered to a patient as a solution in DMSO. As the solubility of the polyurethane material used in the method of the present invention is also improved by the addition of small amounts of DMSO, the inclusion of adriamycin is particularly favorable.

The liposomes and/or therapeutic agents are, in some embodiments, supplied in a constant concentration over the course of embolization, together with the at last one gelling component and/or the gelling agent and/or a separate transport medium.

Preferably, however, the concentration of the liposomes and/or therapeutic agent is varied. Particularly preferably, a higher concentration is supplied at the beginning than at the end of the embolization. This allows supplying a relatively high amount to the tumor at the time of treatment, for a continuous release over time upon degradation of the at least one gelling component, and also for avoiding the spreading of the therapeutic agent or liposome in the opposite direction, i.e. away from the tumor. It is particularly preferred that at the end of the embolization treatment, the at least one gelling component and the gelling agent without the addition of liposomes or therapeutic agent is supplied to the blood vessel, such that the covering regions of polymer away from the tumor to not contain any toxic compounds.

In a further preferred aspect, the present invention also refers to a set comprising a double or multi lumen microcatheter and a polyurethane material as described above. Such a set is ideal for use in the method of the present invention.

Said set will, in some embodiments, further comprise additional double or multi lumen microcatheter(s), allowing for several treatments with fractions of the polyurethane material, and/or one or more therapeutic agents and/or liposomes as described above.

In a preferred embodiment the therapeutic agent is bound to a magnetic nano-based material scaffold. The magnetic nano-based material is able to be used to guide the therapeutic agent with the help of magnetic fields. Therefore, the magnetic fields push and pull the magnetic nano-based material inside of the body while the nano-based material is transported with the bloodstream. As an alternative, the therapeutic agent is able to also be stored in a micro-robot. This micro-robot or Nano robot has a magnetic part and is able to be guided with magnetic fields. The micro-robot is able to either move actively with the help of a mean for transport such as, wheels, a caterpillar or a propeller or move passively with the bloodstream. The term “nano-based material” is here defined as a material of which a single unit is sized (in at least one dimension) between 1 to 1000 nm (10-9 meter).

### Differentiator—Specificity of Tumor Cell Targeting via Surface Receptors, Molecules, and Charges

The nano-based material scaffold, a drug-delivery vehicle, is able to be further functionalized with folic acid as the folate receptor is overexpressed on a large number of tumor cells especially breast, lung, kidney, ovarian, and other epithelial derived cancers16. Furthermore, receptor mediated tumor-targeted drug delivery is gaining traction as a modality to treat solid tumors by capitalizing on receptors overexpressed on tumor cells to achieve focused delivery and accumulation of the pharmaceutical agent in tumor tissues. Such receptors over expressed specifically on tumor cells include folate, growth factor receptors (EGFR, VEGF-R, IGFR), chemokine receptors, hormonal receptors (i.e. estrogen, androgen, and HER-2 receptors to name a few. Functionalizing the drug delivery vehicle with ligands targeting receptors overexpressed on specific cancers combined with the specific localized delivery achievable with the micro-catheter system could provide a major breakthrough in drug penetration and anti-tumor efficacy.

Another method for cancer cell targeting is based on cell surface charge. Cancer cells have been demonstrated to be often characterized by negative surface electrical charge due to unique metabolic processes that occur in cancer cells but not in normal cells, which are generally charge neutral or positively charged. Many anti-cancer drugs are acidic and thus negatively charged as well. The resulting electrostatic repulsion inhibits penetration of the anti-cancer drug into the tumor. However, since cancer cells interact strongly with positively charged materials this may be leveraged diagnostically and therapeutically to target and increase efficiency of therapies.

In one embodiment, the active pharmaceutical ingredient is able to be encapsulated in a drug delivery vehicle and treated with atmospheric cold plasma to impart positive charge and cause the drug delivery vehicle to be attracted to the tumor cells in preference to normal cells. Atmospheric plasma treatment is gaining traction for its therapeutic use in several applications within oncology. An example embodiment includes encapsulation of the active pharmaceutical ingredient in nanoparticles or microparticles with biodegradable biosorbable polymers like poly lactic-co-glycolic acid (PLGA) polymer, followed by plasma treatment to generate an active positive or negative ionic charges in addition to creating chemical fee radicals on the external surface of the particle. These particles may then be embedded in the embolus body by introducing them into the gelling component stream. Alternatively, the particles can be delivered directly to the specific tumor site by the micro catheter. In some cases it may be desired to place a negative charge on the particles if the cancer cells have a positively charged cell surface.

Another embodiment may use bicarbonate to neutralize and protonate the acidic nature of cell-surface of the targeted cancer cells. Bicarbonate is present in the blood as a buffering agent. Previous studies of the use of additional bicarbonate by IV administration to neutralize the negative surface charge of cancer cells showed increased efficacy of the anti-cancer drug through improved penetration into the tumor cells. However, it also improved the penetration of the drug into normal cells thus killing many off-target normal cells. As a result, the use of bicarbonate for charge neutralization is not efficacious at the whole body level. However, local administration of bicarbonate will not have the systemic negative effects produced by whole body introduction of the additional bicarbonate by IV administration.

In various embodiments of the inventive method, bicarbonate may be introduced by one of the lumens of the multi-lumen catheter to neutralize the tumor cell surfaces in the immediate area of the catheter. This local administration of the bicarbonate avoids the negative effects of a systemic administration by IV. Anti-cancer drugs may then be delivered to the same local area via another lumen of the multi-lumen catheter. The drugs will be more effective in penetrating the neutralized membranes of the tumor cells in that area due to the lack of charge repulsion.

In a preferred embodiment of the inventive method, bicarbonate is supplied to the blood vessel, preferably together with the at least one gelling component. The bicarbonate ion will, in some embodiments, protonate an extracellular tumor surface. The bicarbonate is preferably dissolved in an aqueous solution. The concentration of the bicarbonate in the aqueous solution is preferably not too high and the bicarbonate should be present in its ionic form. Further, preferably the bicarbonate is supplied in combination with an emulsifying agent such as poloxamer or with a pump inhibitor such as dexlansoprazole, esomeprazole, pantoprazole and coumarine. The bicarbonate is able to also be supplied in an encapsulated form or by a microcatheter in form of an aqeuous solution or as an emulsion. This lowering of the proton concentration on the outer tumor cell surface thus increases the pH of the tumor cell and provides for a better environment, in particular, for the slightly acidic therapeutic agent. This action could result in a much decreased tumor effluent force hence improved intake of the therapeutic agent inside the tumor cell and significantly better efficacy.

Another mechanism used to deliver anticancer drugs, as well as vaccines and other drugs, are virosome structures. Often the payload drug or molecule is contained in the inner layer of a virosome. These virosomes have the capability to attach and fuse into cells membranes including the membranes of tumor cells. When the artificial virosome, without the infective, viral mRNA normally carried by the virus but instead contain such a therapeutic agent, fuses with the cell membrane of a tumor cell, it releases the therapeutic agent into the tumor cell. This mechanism mimics the normal mechanism of a viral infection. However, the hemagglutinin (HA) protein in the protein coat of the virosome may interact with the sialic acid residues on normal cells, preventing adhesion of the virosome to the cell membrane of cancerous cells. PEG and PEG lipids covering the virosome will reduce and eliminate this reaction hence increasing the strength and effectiveness of the preferential adhesion of the virosome to the tumor cells through antibody redirecting and other similar mechanisms.

In some embodiments of the claimed invention, components of the hydrogel include PEI and PEG. As a result, virosomes carrying the therapeutic agents such as anti-tumor drugs may be incorporated into the in the PEG stream. The resulting hydrogel will contain the virosomes in the matrix of the hydrogel. As the hydrogel degrades the virosomes will be release from the matrix and then perform their therapeutic function over an extended time period. Alternatively, the virosomes may be mixed with PEG and delivered separately into the site of the tumor. More complicated compositions may be formed by varying the concentration of virosomes in the PEG stream during formation of the hydrogel. For example, layers of medicated hydrogel and unmedicated hydrogel may be planned and then formed in situ based on mapping the tumor. First, a layer of the hydrogel can be created from a mixture of PEG and virosomes, then another hydrogel layer without the virosomes may be created by injecting PEG without virosomes through the catheter. Structures such as this allow release of a therapeutic agent and adhesion/fusion of the virosome to the tumor cell membrane.

### Differentiator—Incorporation of Contrast Media Detectable by Xray and MRI Imaging

Preferably the nano-based material scaffold is composed of superparamagnetic iron oxide nanoparticles, which is able to serve dual functions as a scaffold for delivery of therapeutic agents and also as a T2 weighted MRI contrast agent8. Iron based MRI contrast exists in two types: superparamagnetic iron oxide and ultrasmall superparamagnetic iron oxide. These contrast agents consist of suspended colloids of iron oxide nanoparticles and when injected during imaging reduce the T2 signals of absorbing tissues. In combination with platinum, superparamagnetic iron-platinum particles (SIPPs) have been reported and had significantly better T2 relaxivities compared with the more common iron oxide nanoparticles. Other nanoparticles with MRI applications include gold-iron oxide, iron-cobalt nanoparticles. Coating with a layer of polycrystalline Fe3O4 or a graphitic shell enhances stability and makes them better able to provide contrast in MRI imaging19. In contrast to gadolinium-based MRI contrast agents iron based agents are not retained in the brain, and they are metabolized into a soluble and nonsuperparamagnetic form of iron which is incorporated into the normal iron pool within a couple of days19. The combination of leaky vasculature and poor lymphatic drainage in tumors enables the supraparamagnetic iron particles to enter and be retained in solid tumors. Another advantage of an iron based contrast agent is the manipulability by electromagnetic field.

### Experimental Data

### Polyurethane Synthesis

320 ml 1,4 Dioxane (ROTISLOV, Carl Roth) and 64 ml N,N-Dimethylformamid (anhydrous, Sigma Aldrich) were added to a flask. 7.28 g (113 mM) of Hexamethylenediisocyanat (≥99%, Sigma Aldrich) were added. Then, 26.0 g (104 mM) Polytetrahydrofuran (Mn 650, Sigma Aldrich) were added before incubating the mixture for 24 hours at 100° C. 1.337 g DIAZ (Glycerine monoester of Amidotrizo acid) were dissolved in 40 ml 1,4 Dioxane and 10 ml Dimethylformamid. The dissolved DIAZ was added to the polyurethane and incubated for another 24 h at 100° C. The mixture was cooled down, put in a dropping funnel and precipitated with ddH2O. The precipitate was washed twice with ddH2O and let it dry.

### Microcatheter Experiments

14.55 ml EtOH (puriss p. a., ACS reagent, prima fine spirit, without additive, Sigma Aldrich) were mixed with 0.45 ml DMSO (≥99%, Sigma Aldrich) and dissolved 4.5 g polyurethane. Polyurethane solution was extruded through a microcatheter with a 0.4 mm diameter or a 0.6 mm diameter, respectively.

### Experimental Data for Polyethylene Glycol Polyethylene Imine Hydrogel Synthesis

In one embodiment of the inventive method, both PEI and PEG are separately dissolved in PBS buffer solution at different concentrations: 0.332 g/ml PEG and 0.020 g/ml PEI; or 0.498 g/ml PEG and 0.030 g/ml PEI; or 0.664 g/ml PEG and 0.040 g/ml PEI.

In some embodiments, contrast agents, including X-Ray and MRI contrast agents, may be bonded to the backbone of the gel-foam. Such agents include glycerine monoester of diatrizoic acid, a glycerine monoester of a triiodobenzoic acid derivative and an iodinated pyridon-4 derivative, superparamagnetic iron base agents, and gadolinium agents. In various embodiments of the inventive method, the X-ray or MRI contrast agents may be bonded to either the backbone of the PEI component or the PEG component.

The PEI polymer acts as a cross-linker agent, referred to sometimes herein as the gelling agent, inducing the PEG gelation immediately after contact with the PEG solution. The primary amines of the PEI react with the PEG eliminating the Succinimydil groups in the process and forming amide bonds. The two component liquid system of PEI and PEG solutions is them ready to deliver to target artery via the micro-catheter system.

In some embodiments, selective therapeutic agents for localizing delivery can be incorporated into one or both of the component solutions prior to application to the artery or tumor. These therapeutic agents in the PEG and PEI components of the gel become an integral part of the hydrogel and will be released upon degradation of the hydrogel. Examples of therapeutic agents include liposomes, virosome, micro/nanospheres, Peptides, Proteins, nanorobotics systems, bicarbonate tumor cell neutralizing agents, or other therapeutic agents.

In some embodiments, the hydrogel delivery is performed through a concentrical multi-lumen microfluidic catheter containing at the end a microfluidic mixing chamber. In varying embodiments, the flow rate ratio through the two lumens in the catheter is 1:1. In the mixing chamber, PEI and PEG react, allowing the cross-linked hydrogel's immediate formation, which is finally ejected to the desired location. In a preferred embodiment, the PEG solution flows through the inner lumen, while the PIE flows through the outer lumen.

In some embodiments of the micro-catheter system the multi-lumen catheter includes an inner tube having an outer diameter of 0.254 mm, and an outer tube having an outer diameter of 0.3048 mm. In some embodiments the mixing chamber has a length of 2 cm. In a preferred embodiment the mixing chamber is a microfluidic mixing chamber.

“Substantially” means to be more-or-less conforming to the particular dimension, range, shape, concept, or other aspect modified by the term, such that a feature or component need not conform exactly. For example, a “substantially cylindrical” object means that the object resembles a cylinder but may have one or more deviations from a true cylinder.

“Comprising,” “including,” and “having” (and conjugations thereof) are used interchangeably to mean including but not necessarily limited to, and are open-ended terms not intended to exclude additional, unrecited elements or method steps.

Changes may be made in the above methods, devices and structures without departing from the scope hereof. Many different arrangements of the various components depicted, as well as components not shown, are possible without departing from the spirit and scope of the present invention. Embodiments of the present invention have been described with the intent to be illustrative and exemplary of the invention, rather than restrictive or limiting of the scope thereof. Alternative embodiments will become apparent to those skilled in the art that do not depart from its scope. Specific structural and functional details disclosed herein are not to be interpreted as limiting, but merely as a representative basis for teaching one of skill in the art to employ the present invention in any appropriately detailed structure. A skilled artisan may develop alternative means of implementing the aforementioned improvements without departing from the scope of the present invention.

It will be understood that certain features and subcombinations are of utility and may be employed without reference to other features and subcombinations and are contemplated within the scope of the claims. Not all steps listed in the various figures need be carried out in the specific order described.

1. A method for blood vessel embolization in a blood vessel, comprising the steps of:
supplying at least one gelling component in liquid form to the lumen of a blood vessel by means of a microcatheter having a first lumen, a second lumen, and a mixing chamber, and
wherein the at least one gelling component is biocompatible and biodegradable; and
wherein the at least one gelling component forms in situ a deformable solid matrix in the lumen of the blood vessel.

2. The method of claim 1 wherein the first lumen is at least partially disposed inside the second lumen.

3. The method of claim 2 wherein the mixing chamber is formed by a section of the second lumen that extends beyond an end of the first lumen.

4. The method of claim 2 wherein a first gelling component of the at least one gelling component is supplied through the first lumen and a second gelling component of the at least one gelling component is supplied through the second lumen.

5. The method of claim 1 wherein the first gelling component comprises a composition selected from the group consisting of a first polyurethane precursor or a first hydrogel precursor; and
wherein the second gelling component comprises a composition selected from the group consisting of a second polyurethane precursor or a second hydrogel precursor; and
wherein the one of the at least one gelling component is bonded to a contrast material to facilitate imaging capabilities.

6. The method of claim 5 wherein the contrast material is selected from the group consisting of superparamagnetic iron oxide nanoparticles, derivatives of glycerine monoester of diatrizoic acid, a glycerine monoester of a triiodobenzoic acid derivative, and an iodinated pyridon-4 derivative.

7. The method of claim 5 where the polyurethane precursor is selected from the group consisting of a diol precursor compound and a diisocyanate precursor compound.

8. The method of claim 5, wherein a first compound of the at least one diol precursor compound is selected from the group consisting of a glycerine monoester of diatrizoic acid (1), a glycerine monoester of a triiodobenzoic acid derivative (2, 3, 4, 5), and an iodinated pyridon-4 derivative (6):

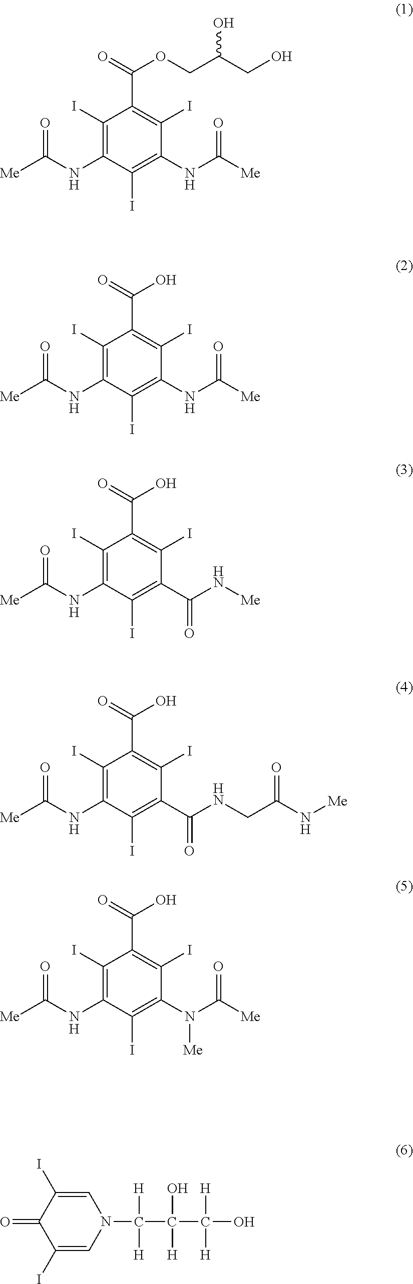 [SMILES: CC(=O)Nc1c(I)c(NC(C)=O)c(I)c(C(=O)OCC(O)CO)c1I ; CC(=O)Nc1c(I)c(NC(C)=O)c(I)c(C(=O)O)c1I ; CNC(=O)c1c(I)c(NC(C)=O)c(I)c(C(=O)O)c1I ; CNC(=O)CNC(=O)c1c(I)c(NC(C)=O)c(I)c(C(=O)O)c1I ; CC(=O)Nc1c(I)c(C(=O)O)c(I)c(N(C)C(C)=O)c1I ; O=c1c(I)cn(CC(O)CO)cc1I]

9. The method of claim 8, further comprising a second compound of the at least one diol precursor compound, wherein the second compound is selected to be co-condensible with the first compound, and is selected from the group consisting of:
a. bi-valent alcohols of the type
HO—(CH₂)ₙ—OH n=2-12
b. polyether diols of the type 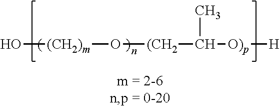
c. polyester diols on the basis of:
adipic acid/ethylene glycol-co-propylene glycol, adipic acid/ethylene glycol, adipic acid/propylene glycol;
I. polyglycol diol and polyglycol-co-lactide diol;
II. poly-3-hydroxybuyric acid diol and poly-3-hydroxybutyric acid-co-3-hydroxy valeric acid diol;
III. poly-3-hydroxy valeric acid diol;
IV. poly-caprolactone diol;
V. de-polymerized cellulose;
VI. de-polymerized cellulose acetate.

10. The method according to claim 9, wherein the polyester diols on the basis of II, III and IV are produced by transesterification of higher molecular polyesters with ethylene glycol, diethylene glycol and triethylene glycol with simultaneous cleavage into a plurality of macro-diols having a mean molecular weight between about Mn 500 and 10,000.

11. The method according to claim 7, wherein a first compound of the at least one diisocyanate precursor compound is selected from the group consisting of:
a. 5-isocyanato-1-(isocyanatemethyl)-1,3,3-tri-methylcyclohexane;
b. 1,3-bis(1-isocyanato-1-methyl)benzene;
c. hexamethylene diisocyanate; and
d. 2,2,4-trimethyl-hexamethylene diisocyanate.

12. The method of claim 5 wherein the polyurethane material is represented by the formula (7): 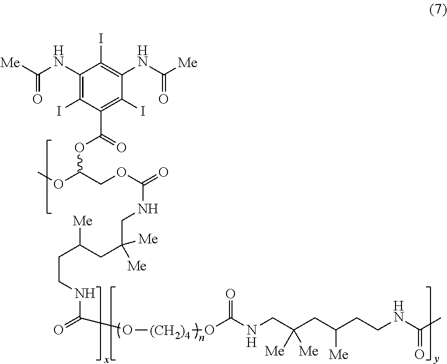
where x≥1, y≤1000, and 1≤n≤50.

13. The method according to claim 1 wherein the first lumen is completely surrounded by the second lumen.

14. The method of claim 1 wherein the second gelling component is supplied to the blood vessel before the first gelling component.

15. The method of claim 14 wherein during a release of the first gelling component there is also a release of the second gelling component.

16. The method of claim 15 wherein the second gelling component consists of at least 50% water or blood.

17. The method of claim 2, wherein the at least one gelling component is a polyurethane material and is provided in the form of an ethanolic solution, suspension or emulsion.

18. The method of claim 1, further comprising the step of supplying one or more liposomes, virosomes, micro/nano spheres, peptides, proteins, nanorobotics systems, tumor cell bicarbonate neutralizing agents, or therapeutic agents to the blood vessel through the microcatheter.

19. The method of claim 18, where in the one or more liposomes, virosomes, micro/nano spheres, Peptides, proteins, nanorobotics systems, tumor cell bicarbonate neutralizing agents, or therapeutic agents are supplied together with a gelling component of the at least one gelling component.

20. The method of claim 18 wherein the therapeutic agents are selected from the group consisting of anti-neoplastic agents falling into the broad classes of cytotoxic chemotherapy, small molecule targeted agents, biologics, anti-viral agents, or nucleotide based therapeutics.

21. The method of claim 18, wherein the one or more therapeutic agents are selected from the group consisting of alkylating agents such as cisplatin (DDP) carboplatin (CBP), and oxaliplatin (L-OHP), nitrogen mustard, chlorambucil, cyclophosphamide (CTX), and ifosfamide (IFO), nitrosureas, such as N-methyl-N-nitrosurea (MNU), N′-[(4-amino-2-methylpyrimidin-5-yl)methyl]-N-(2-chloroethyl)-N-nitrosourea (ACNU), 1,3-bis(2-chloroethyl)-1-nitrosourea (BCNU), N-(2-chloroethyl)-N′-cyclohexyl-N-nitrosourea (CCNU), and N-(2-chloroethyl)-N′-(4-methylcyclohexyl)-N-nitrosourea (methyl CCNU); ethylenimines, such as 2,4,6-triethylene melamine compound (TEM) and thiotepa; methane sulfonates, such as busulfan, dacarbazine, procarbazine, plant alkaloids and anti-microtubule agents including vinblastine (VLB), vincristine (VCR), vindesine (VDS), navelbine (NVB), paclitaxel (PTX), and taxotere, anti-tumor antibiotics (intercalating agents) including anthracycline antitumor antibiotics, such as adriamycin (ADM; doxorubicin), daunorubicin (DNR), epirubicin (EPI or E-ADM), mitoxantrone (MTT, DHAD), and pirarubicin (THP); actinomycin antitumor antibiotics, such as actinomycin D (ACD); bleomycin antitumor antibiotics, such as bleomycin and pingyangmycin (A5); mitomycin antitumor antibiotics, such as mitomycin A, mitomycin B, and mitomycin C (MMC); mithramycin antitumor antibiotics, such a mithramycin (MTH) and olivomycin, other antibiotics, such as streptozotocin (STT), antimetabolites including thymidylate synthase inhibitors, such as 5-fluorouracil (5-FU), ftorafur (FT-207), tegadifur (difuradin FD-1), tegafur-uracil (UFT), and furtulon (5-DFUR); dihydrofolate reductase inhibitors, such as methotrexate (MTX), DNA polymerase inhibitors, such as cytarabine (Ara-c); ribonucleotide reductase inhibitors, such as hydroxyurea (HU), inosine dialdehyde, adenosine dialdehyde, and guanazole; purine nucleotide synthesis inhibitors, such as 6-mercaptopurine (6-MP) and andaminopterin, topoisomerase inhibitors, small molecule targeted therapies including but not limited to tyrosine kinase inhibitors such as imatinib, erlotinib, sunitinib, gefitinib, sorafenib, dasatinib, lapatinib, and nilotinib and the like, biologic therapies, including but not limited to such as ipilimumab, nivolumab, pembrolizumab and the like, and anti-viral drugs such as protease inhibitors, nucleotide/non-nucleotide polymerase inhibitors and interferons.

22. The method of claim 18, wherein the concentration of liposomes, virosomes, micro/nano spheres, Peptides, proteins, nanorobotics systems, tumor cell bicarbonate neutralizing agents, or therapeutic agents is varied during the supply thereof to the blood vessel.

23. The method of claim 22 wherein the concentration of liposomes or therapeutic agents is highest at the beginning of the supply thereof.

24. The method of claim 18 wherein the one or more therapeutic agent is bound to a magnetic nano-based material scaffold.

25. The method of claim 24 wherein the magnetic nano-based material scaffold is supraparamagnetic iron oxide that also functions as an MRI contrast agent.

26. The method of claim 24 wherein the magnetic nano-based material further comprises a ligand targeting receptors for a receptor that is overexpressed by a targeted cancer cell, including but not limited to folate, growth factor receptors (EGFR, VEGF-R, IGFR), chemokine receptors, hormonal receptors (i.e. estrogen, androgen, and HER-2 receptors.

27. The method of claim 1 further comprising the step of supplying an MRI contrast agent to the blood vessel together with a gelling component of the at least one gelling component.

28. The method of claim 27 wherein the MRI contrast agent is an iron-based contrast agent stabilized by incorporation of platinum.

29. The method of claim 27 wherein the MRI contrast agent is an iron-based contrast agent stabilized by coating with a layer of polycrystalline Fe3O4 or a graphitic shell.

30. The method of claim 1 further comprising the step of supplying bicarbonate to the blood vessel together with a gelling component of the at least one gelling component.

31. The method of claim 1 wherein the microcatheter is a catheter for radiofrequency ablation that includes a plurality of variable stiffness segments and a magnetic tip.

32. The method of claim 31 wherein the variable stiffness segments comprise a low melting point alloy and the magnetic tip of the catheter is controlled by an external magnetic field.

33. The method of claim 32 wherein the magnetic field has a magnetic gradient of at least 0.1 T/m.

34. The method of claim 5 wherein the first and second hydrogel precursors comprise dextran and chitosan.

35. The method of claim 18 where the liposome is a virosome having a virosome membrane that incorporates PEG lipids.

36. The method of claim 2 wherein the mixing chamber is a microfluidic mixing chamber.

37. The method of claim 5 wherein the first gelling component comprises a polyethylene glycol and the second gelling component comprises a polyethylene imine.

38. The method of claim 37 wherein the first gelling component comprises an activated polyethylene glycol.

39. The method pf claim 38 wherein the activated polyethylene glycol is PEG Succinimydil Propionate (SPA- PEG-SPA) of 3.4K.

40. The method of claim 37 wherein the polyethylene imine is a branched polyethylene imine of 2K.

41. The method of claim 18 wherein the virosomes are specifically added to the PEG stream.

42. The method of claim 1 wherein the at least one gelling component is biocompatible, biodegradable, and contains a radiopaque element.

43. The method of claim 1 wherein the at least one gelling component comprises a polyurethane precursor or a PEI/PEG hydrogel.
